(19) United States
(12) Patent Application Publication
(10) Pub. No.: US 2007/0053856 A1
(43) Pub. Date: Mar. 8, 2007

(54) Integrating ultraviolet exposure detection devices

(76) Inventors: Hans O. Ribi, Hillsborough, CA (US); David A. Frankel, Kirkwood, MO (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 11/513,899

(22) Filed: Aug. 30, 2006

Related U.S. Application Data
(63) Continuation of application No. 11/223,446, filed on Sep. 9, 2005, now abandoned.
Application No. 11/223,446 is a continuation of application No. 10/272,251, filed on Oct. 15, 2002, now abandoned.
Application No. 10/272,251 is a continuation-in-part of application No. 09/539,487, filed on Mar. 30, 2000, now Pat. No. 6,465,791.
Application No. 09/539,487 is a continuation of application No. 09/016,683, filed on Jan. 30, 1998, now Pat. No. 6,046,455.

(51) Int. Cl. A61K 8/00

(52) U.S. Cl. 424/61; 424/59; 424/70.1

(57) ABSTRACT

Devices or formulations incorporating photochromic agents and methods of using them are provided. The compositions are useful in connection with personal care, light and/or ultraviolet radiation detection, imaging and printing applications.

### CROSS-REFERENCE TO RELATED APPLICATIONS

This application is a continuation in part of U.S. Ser. No. 09/539,487 filed Mar. 3, 2000, now U.S. Pat. No. 6,465,791, issued Oct. 15, 2002, which is a continuation of U.S. Ser. No. 09/016,683, filed Jan. 30, 1998, now U.S. Pat. No. 6,046,455, issued Apr. 4, 2000, which disclosures are hereby incorporated by reference.

### INTRODUCTION

1. Field of the Invention

The invention relates to compositions that reversibly or irreversibly change color in response to light/UV radiation and methods of using them. The invention is exemplified by the use of diacetylenic compounds in detection and measurement of light/UV exposure of a substrate and in development of graphics and messages upon exposure to light/UV radiation.

2. Background

While exposure to sunlight may have some benefits, particularly the healthy appearance of a tan, ultraviolet radiation has substantial detrimental effects. The ultraviolet (UV) radiation can cause rapid aging and hardening of the skin, much like the tanning of leather. In addition, the ultraviolet radiation can cause severe erythmia, which in severe cases can be physically debilitating. Of particular concern, the ultraviolet radiation can cause DNA damage, which can lead to skin cancer or other cellular proliferative diseases. Skin cancer is the most prevalent of all cancers and is among the most preventable forms of cancer. Basal cell carcinoma is very common among those with fair skin and hair, but while rarely metastasizing can spread to bone. Malignant melanoma develops on the skin of about 35 thousand Americans annually, resulting in about 7 thousand annual deaths. Finally, squamous cell carcinoma, which is found on the ear, face, lips and mouth, is the second most common skin cancer in Caucasians, resulting in about 2.3 thousand deaths annually. UV radiation can also result in severe eye damage, leading to corneal burns, retinal burns, pingueculae and pterygium, cortical cataracts and macular degeneration. Cataracts are the major cause of visual impairment, with UV exposure being among the leading causes. Worldwide, 17 million people are blinded by cataracts, with nearly 1.5 million cataract extractions being performed annually in the United States.

Children, elderly, immunocompromised individuals, individuals with skin disorders, e.g. lupus erythematous patients, or others particularly susceptible or sensitive to ultraviolet radiation are particularly vulnerable to the injuries and disorders resulting from UV radiation.

These susceptible individuals should have the capability of being warned about overexposure. In many cases, individuals rely on a suntanning screen which absorbs ultraviolet light. However, these screens and lotions are lost over time due to sweat, abrasion, exposure to water, and the like. Under these circumstances, the protection is lost and the person is subjected to unwanted ultraviolet radiation. UV protective coatings are available for eye ware. However, individuals have no convenient way of knowing the level of protection they are receiving. Also, UV lens coatings cannot protect against stray light which enters around the glasses.

The interaction of either sunlight or artificial light with various objects can have many additional consequences. For example, poop or medicine may deteriorate by exposure to light or ultraviolet radiation. Also, each plant species typically grows best under certain lighting conditions.

There is a need for practical UV detection devices, which can be conveniently used and carried, so that they are available when the need arises.

Relevant Literature

A number of photochemical systems have been described in the literature to act as dosimeters for ultraviolet light. See for example, U.S. Pat. Nos. 5,581,090; 5,028,792; 3,710,115 and references cited therein. Also, devices have been made available, such as a card, UV Card, available from the South Seas Trading Co., and a UV meter, called SafeSun, available from Online Catalog.

### SUMMARY OF THE INVENTION

Devices and compositions that include a photochromic agent which changes color upon exposure to ultraviolet light are provided tgether with methods of using them. Particularly, diacetylenes are used in a form that adheres to a substrate or admixes with a liquid formulation thus generating many novel properties. The methods include combining the photochromic agent with a substrate and exposing the substrate to light and/or ultraviolet radoatopm to alter the color of the photochromic agent. The compositions are useful in connection with personal care products, light or ultraviolet radiation detection, imaging, and printing applications.

### DESCRIPTION OF THE SPECIFIC EMBODIMENTS

Compositions and methods are provided for detection and use of ultraviolet or light radiation for a variety of applications using photochromic agents that can be either reversible or irreversible depending upon the intended application. Of particular interest is admixing the compositions with personal care items such as sunscreens, cosmetics, hair products and nail products, particularly for detection of cumulative ultraviolet radiation exposure levels and/or for providing color or for enhancing the colors of existing dyes. The measurements of light and/or ultraviolet radiation cumulative exposure also are useful for applications such as determining the optimal location for a particular plant or plant type and for monitoring total UV exposure of products such as drugs that decay upon exposure to UV radiation. Also of interest is use of the compositions for imaging for example from a computer screen and for printing applications both whimsical and useful. Whimsical applications include generation of images, particularly in liquids. Useful applications include generation of messages on a substrate indicating the cumulative amount of light/UV exposure of an article or person.

In some embodiments subject compositions are used as or in connection with inks. Depending on the specific application, they may be printed on substrates either as a design or as a uniform coating. The color of the printed ink changes as a result of exposure to radiation. For this reason, it is often desirable to cover the printed substrate with a protective layer that eliminates exposure to radiation.

Printed materials are often desirable for packaging. Promotional messages can be printed on packaging materials, and they can become visible or change color after opening. Inks comprising the subject compositions may be used for printing any text or graphics. Of particular interest are inks that photobleach as a result of continued exposure to radiation. Such inks are useful in manufacturing printed materials with a limited life time.

The subject compositions are also useful for manufacturing devices for measuring the extent of radiation exposure. Both time of exposure and radiation intensity correlate with the rate of color change of the photochromic agents. Therefore, color development can be calibrated to correspond to predetermined levels of radiation. Radiation measurement finds many applications, including monitoring indoor or outdoor activities, pharmaceutical or medical packaging, and determining light exposure levels for vegetation and planting.

In addition, photochromic agents for detection of radiation can be integrated with other type of color indicators. In some embodiments, the photochromic agents are also capable of indicating by color changes strain, temperature, friction or the solvent present. Alternatively, the photochromic agents may be combined with silicon rubber compositions to create thermo-responsive or photo-color developing compositions.

The subject photochromic agents ate also used in a liquid phase in some embodiments. Liquids comprising these compositions have the capacity of maintaining transient images that are formed in response to radiation. These images may be formed, for example, using a UV protective mask on a wall of the fluid containing vessel.

In some imaging application embodiments, the photochromic agent is coupled to a light isomerising compound. The resulting material may be used, for example, to create integrating light or UV induced graphic changes where the changes result in a visible graphic transformation from one image to another. Self-transitioning graphics find use as two-dimensional graphic without the use of optical interference or prisms used in holograms. In other imaging application embodiments, the subject photochromic compositions are useful in obtaining a printed image of a screen. For example, a substrate can be coated with a photochromic composition is attached to a screen displaying an image. The substrate is then detached an laminated so as to prevent further exposure to light.

For the elastic bands, any elastomeric material which is not irritating to the skin and compatible with the photochromic agent may be employed. The elastomer will be substantially transparent or of sufficiently small dimension as not to leave a readily observable band upon tanning. Elastomers include: polyisobutylene, ethylenepropylene copolymers, polyisoprenes, polybutadienes, etc. Elastomers may be selected to absorb any sun tan protective coatings, much as the skin will absorb the coatings to varying degrees. These elastomers may be formed in bands or ribbons, generally having thicknesses of about 0.5 mm to about 5 mm, using not more than about 3 mm and may have the same width or be wider, depending upon the design and shape of the band. The band may be continuous or be joined by a link or clasp. If desired, a UV opaque cover may be employed, particularly one which is scored. By having the cover scored, individual portions of the cover may be removed, so that a single band may be used repeatedly, until the entire band has been exposed and exhausted. The cover may be paper or plastic, which will be able to withstand the conditions under which it is used. The band size or diameter is selected to fit snugly around a body part, such as a finger, wrist, forearm, bicep, ankle, or the like.

The elastomeric device may be preformed and combined with the photochromic agent, e.g. impregnated, painted, coated, sprayed, etc., or may be formulated with the elastomeric material and appropriately molded, extruded, cast, etc. The elastomeric device can be preformed, followed by impregnation with a solution of the photochromic agent, conveniently in combination with a polymer which aids in the coating and/or entry of the photochromic agent and retention in the elastomer. Polymers which may be employed include acrylic and styrene polymers, rubbers, and the like. Various solvents may be employed which will soften, but not dissolve, the elastomer and allow for impregnation of the elastomer with the photochromic agent. The solvent which is selected should be volatile, having a boiling point at ambient pressure above ambient temperature and less than about 100.degree. C. and residues of the solvent should not be irritating to the skin. The solvent will soften the surface of the elastomer or may impregnate the elastomer to a portion of the thickness of the elastomer, but will normally not dissolve the elastomer, so that upon volatilization of the solvent, the elastomeric device will be in substantially the same form as originally. When coating the elastomeric surface, the coating solvent will usually provide for impregnation of the photochromic agent into the elastomer or provide a polymeric protective coating which retains the photochromic agent on the surface of the elastomeric device. Illustrative solvents include: chloroform, diethyl ether, ethyl acetate, butanone, tetrahydrofuran, toluene, dichloromethane and the like. The concentration of the photochromic agent will be sufficient to provide a visual change in color of the elastomer upon exposure to UV radiation and may vary depending upon the nature of the elastomer, the photochromic agent, and the like. Generally, the concentration would be in the range of about 10 to 500 mg/ml, preferably from about 50 to 250 mg/ml. If a polymeric agent is also present, this may be present in from about 10 to 500 mg/ml, more usually from about 50 to 200 mg/ml. Conveniently, various addition or condensation polymers may be used, such as acrylic, vinyl, polyalkylene, polyesters, polyether polymers.

After impregnation, the solvent will be evaporated, using vacuum, elevated temperatures, ambient conditions, or the like. During the treatment and thereafter during storage, the elastomeric devices should be protected from ultraviolet light, conveniently by being stored in an opaque container which does not transmit ultraviolet light or covered with an opaque cover.

One can provide for determining gradations of irradiation, by using varying amounts of the photochromic agent. Thus, by varying the amounts within the range indicated previously, one can provide for detecting different levels of exposure. For example, one may have a series of formulations with 50 mg/ml, 100 mg/ml, and 200 mg/ml, where the formulations are present at different sites of the device, where each will be exposed to the same amount of UV radiation. The darkness of the color generated by exposing the photochromic agent to irradiation can also be used to judge the amount of irradiation. Depending on the structure of the device, the photochromic agent may be present as a line, band, design, message, two-dimensional figure, or the like.

Instead of an elastomer, a non-elastomeric substrate, such as a tape, decal, label, plastic bottles, shrink wrapped plastic labels, and paper may be employed. The film is or has an adhesive suitable for adhering to the epidermis, including fingernails. Except for those epidermal areas which are not readily affected by UV radiation, e.g. fingernails, the film will be transparent. The film may be combined with the photochromic agent in a variety of ways. The film may be impregnated with the solution of the photochromic agent as described above. Alternatively, the photochromic agent may be applied as an ink to the surface of the film, whereby the photochromic agent becomes bound to the film. Various permanent inks or ink matrices which are commercially available may be employed, where the photochromic agent will be mixed with the ink, which ink may be clear (free of other dyes) or be colored with a color that does not interfere with the color development of the photochromic agent. The ink may be painted on to the film substrate by any convenient means, coating, spraying, printing or the like. The concentration of the photochromic agent for impregnation or coating will generally be about the same as the concentration range described above for the elastomer treating solutions.

Adherent devices will normally be adhered to a layer from which the adherent layer may be readily removed while still retaining the adhering layer to the photochromic agent containing film. A wide variety of protective layers are available, as exemplified with Bandaids.RTM., label or decal materials, medical grade tapes, layers of adhesive tape, and the like. Alternatively, one can use an adhesive which can be separated from the surface of the device, when the device is in a roll.

The tapes may take a variety of forms, being opaque, such as adhesive tapes used for covering injuries, or clear, such as novelty tapes. A number of adhesives may be used which have acceptance in other situations, where the adhesive will strongly adhere to the substrate surface, but the tape may be removed by pulling the tape away from the surface.

The tapes may come in a variety of sizes, being relatively small. The clear tape is preferred to allow for a more even tanning of the skin and minimally intrusive appearance.

Usually tapes will vary in length from about 0.5 to 3 cm and in width from about 0.2 to 2 cm. The thickness of the layer containing the photochromic agent will be sufficient to allow the color change to be readily detected within the context of its use. Depending on the concentration of the photochromic agent, the required thickness will vary, generally varying in the range of about 0.1 to 1000.mu., more usually 10-100.mu.

Alternatively, the film may be a decal, which may be applied to the skin, where the decal will usually be at least partially colored so that its presence can be readily detected. The decal will be a preformed film, which will adhere to the skin when pressed against the skin, much as the adhesive tape. In some instances, the decal may require wetting before applying the decal to the skin. It may take a variety of geometric or odd shapes, being in the shape of figures, such as animals, objects, etc., where portions may be colored with a design, which design may be augmented by the development of the photochromic agent. The photochromic agent may result in a design, where the design may have different concentrations of the photochromic agent, so that the design will change as increasing areas of the design become developed. In this way a range of exposure to UV radiation may be determined. Alternatively, the agent may be printed adjacent to colored reference zones which indicate the degree of exposure compared to the darkness of the agent. These zones may be very small, so as to minimize the variation in tan or may be selected to provide a message which the wearer desires.

By virtue of the color change of the photochromic agent, various messages may be printed. By having a contrast between the region in which the photochromic agent is present and the remaining background, a message can be provided related to the status of the UV radiation or other information. Thus, one could print letters with the photochromic agent and provide a background the same as the photochromic agent, but not the same as when the photochromic agent has undergone a color change. Alternatively, one can provide that the message disappears, by having the background similar to the photochromic agent when it has became colored. One can also provide various colors as standards, as the region of the photochromic agent deepens in color. The standards can be related to various levels of UV radiation as described above. When using areas of different concentrations of the photochromic agent, one can provide for almost continuous gradations of UV radiation exposure, between the different concentrations and different standard colors. Transparent coatings or films that block UV light to varying degrees may be placed over areas of the photochromic agent to give differential sensitivities to exposure. These layers may be clear plastic films or clear glasses and may incorporate SPFs or other UV screening compounds.

The devices should have retentivity of the various protective sun coatings, so as to mimic the exposure of the skin. To that extent, the sun screens which are used should be compatible with the device. The film may be porous or non-porous to absorb the sun screen to an acceptable depth. The film should be wet by the sun screen in an analogous manner to the skin. Various plastics may be modified to provide the desired properties, such as chemical or physical modification, use of specific monomers to modify the characteristics of the bulk monomer, or thin coatings of a layer compatible with both the underlying substrate and the sun screen formulation.

One may also provide for various formulations which provide adherent coatings to a body surface, such as the skin, fingernails, or the like. For this purpose, one may use formulations such as fingernail polish formulations, e.g. acrylic paints, combined with the photochromic agent where the nail polish may be clear or colored with a color which does not interfere with the detection of the photochromic agent. A thin layer of the fingernail polish may be coated on one or more fingernails, so that upon UV exposure, the fingernails will turn colored. Alternatively, a hydrophobic composition may be employed, such as a balm, lotion, or the like, where various wax based formulations may be employed having from about 50 to 99 weight percent of a hydrophobic agent, e. g. hydrocarbons, hydrogenated oils, fatty acid esters, etc. Various additives may be included for texture, organoleptic properties, stability, or the like. These include such conventional additives as liposomes, polymerized liposomes, plastic microparticles, protein mixtures, cosmetic formulations and those used in balms.

The photochromic agent may be formulated with a sunscreen lotion as an emulsion, where the photochromic agent is encapsulated in droplets, including liposomes, which break down on spreading on the skin. The breakdown of the droplets results in a strongly adhering film, which will adhere to the skin under the UV radiation protective agent. Various polymers described previously, including celluloses, may be employed in the droplets. Exemplary formulations are described in U.S. Pat. Nos. 5,543,136; 4,897,259; 4,184,978 and 3,895,104. Alternatively, the photochromic agent can be an integral part of the liposome (e.g. diacetylenic lipid), such that the liposome can be both part of the liposome matrix and be the indicator of interest.

The photochromic agent may be readily mixed with the formulation to provide a stick, paint or lotion which formulation when applied to the skin will permit the photochromic agent to strongly adhere to the skin.

In each of these cases, the method of applying the photochromic agent and the base it is mixed with allows for its strong adherence and ready removal of the photochromic agent containing product by convenient means, well known in the literature. Nail polishes may be removed by gentle scrapping or with nail polish remover, balms, e.g. ChapStick.RTM., and other coatings with soap, tapes and decals by pulling off or scraping, or the like.

The photochromic agents of the subject invention are diynes (conjugated diacetylenes), particularly acid, ester, urethane, amide, nitrile, and alcohol monomers of at least about 8 carbon atoms, and not more than about 36 carbon atoms, more usually from about 12 to 30 carbon atoms. The acetylenic groups will generally be displaced from the terminal carbon atoms by at least 1 carbon atom. Various derivatives of the functional groups of the diynes can serve to modify the properties of the diynes for use in a particular formulation. For further information concerning these compounds, see U.S. Pat. Nos. 5,685,641; 5,622,872; 5,618,735 and references cited therein, whose disclosures are herein incorporated by reference as to their more complete description of the monomers and their preparation and derivatives. These compounds are readily formulated as the monomers with a wide variety of formulations, without special requirements, except that they be protected from ultraviolet radiation. They can be dissolved in a wide variety of solvents, mixed with various polymers, and incorporated with different agents, so as to provide for stably dispersed compositions. Upon exposure to ultraviolet radiation, the compositions turn light blue to a black metallic luster.

The subject devices, particularly as clear adhesive films, find use to protect the eye against UV radiation, by using the film in conjunction with the lense of eyeglasses. In this case, one may determine the extent to which the eye has been exposed to ultraviolet light which has come through the lens and stray light which enters from outside the eyeglasses. Conveniently, the sensing film may be placed on the inside of the lens of the glasses, so that the wearer may be able to judge instantly or continuously the level of sun exposure within the eye glass-eye cavity. A light blue color will begin to appear upon mild sun exposure, while the color would deepen upon prolonged or more intense exposure. In addition, as discussed previously areas of different concentration of the photochromic agent may be present, so as to provide a graduation of exposure. Various films may be employed which are clear, such as acrylics, polyalkylenes, polyvinyl ethers and esters, etc., which can be coated with a clear adherent layer. The film may then be adhered to a corner of the lens and monitored during exposure to UV radiation.

The following examples are offered by way of illustration and not by way of limitation.

### EXAMPLES

### Example 1

### Elastic Band Sensors for General Wear

Elastic band sensors were made with either rubber bands (2 inch in diameter, ¼ inch wide, and 1/32 inch thick rubber bands from Alliance Rubber Bands, USA, U.S. Pat. No. 3,787,552) or thin elastic stretch cord (standard white fabric shock cord 1/16 inch thick and 2 inch in diameter tied to create a poop). Ten bands were added to a 100 ml screw cap jar containing 50 ml of a monomer solution (150 mg/ml 10,12-tricosadiyneoic acid in chloroform and 100 mg/ml V825 acrylic plastic) and agitated for 1 minute at room temperature. The bands were conveniently looped through a wire prior to dipping so that they could all be added, removed, and dried simultaneously. The bands were lifted from the solution, shaken over the jar to remove excess solvent, and air dried at room temperature for 3 hours to ensure that all solvent was evaporated. Upon drying, the elastic bands were ready for use as sun irradiation sensors by placing the band around the wrist or ankle and worn during normal activity in the sun.

### Example 2

### Elastic Band Sun Sensors for Finger Ware

Elastic band sensors were made with either rubber bands (¾ inch in diameter, 1/16 inch wide, and 1/32 inch thick rubber bands from Goody Product, Inc.) or thin elastic stretch cord (standard white fabric shock cord 1/16 inch thick and ¾ inch in diameter tied to create a loop). Ten bands were added to a 100 ml screw cap jar containing 50 ml of a monomer solution (150 mg/ml 10,12-tricosadiyneoic acid in dichloromethane) and agitated for 1 minute at room temperature. The bands were conveniently looped through a wire prior to dipping so that they could all be added, removed, and dried simultaneously. The bands were lifted from the solution, shaken over the jar to remove excess solvent, and air dried at room temperature for 3 hours to ensure that all solvent was evaporated. Upon drying, the elastic bands were ready for use as sun irradiation sensors by placing the band around a finger and worn during normal activity in the sun.

### Example 3

### Medical Tape Sun Sensors for General Placement

A 10 foot roll stock medical tape (¼ inch wide transparent easy-tear medical grade) plastic tape was decorated with a 1/16 inch wide line along its length with an ink line containing the monomer 10,12-tricosadiyneoic acid (TDA). The ink line was made with a pen (orange Sharpie brand permanent ink pen). The pen cartridge was removed and the orange ink placed in a 4 dram vial. TDA was added to the ink volume to bring the final concentration to 75 mg TDA/ml ink. The TDA ink mixture was added back to the pen cartridge and replaced in the pen. The tape was mounted on a spool mechanism such that the pen tip made an orange line along the non-adhesive side of the tape as the tape was spooled from one roller to another. The tape was spooled at a rate which allowed the ink to dry prior to overlap between layers. Convenient, ready-to-use sun sensor strips were made by tearing a small piece of tape and applying the piece to skin or clothing. Upon prolonged sun exposure, the orange line turned to a dark black-blue line indicating a high level of UV irradiation.

Tape sensor strips could also be made with dual lines on the tape where one line was made using an orange pen without TDA monomer and the parallel adjacent line was made with TDA monomer. This configuration provided for a tape strip with an orange reference color for an individual for color change comparison.

### Example 5

### Balm Stick for Application to Skin and Other Surfaces

A solid wax-like stick was made using a melted mixture of 44% petrolatums, 1.5% Padimate 0.1% lanolin, 1% isopropyl myristate, and 0.5% cetyl alcohol extracted from a stick of ChapStick.TM. Three percent by weight 10,12-pentacosadiyneoic acid was added and melted into the mixture. The component mixture was heated to a liquid form (greater than 150.degree. F.) in a glass vessel, mixed by stirring, and poured into a plastic cylinder/dispenser with a screw crank at one end. The plastic dispenser was 2 inches long and ½ inch in diameter with a protective cap at its open end. The mixture was allowed to cool at room temperature for 1 hour.

The sun sensor balm could be readily applied to any skin surface, finger nails, or other body part intended to be exposed to sunlight. Upon coating, the balm leaves an invisible film. A light blue color begins to appear upon mild sun exposure. A deep dark blue color appears upon prolonged exposure. The balm can be conveniently washed off using mild soap or wiped off using a napkin.

### Example 6

### Acrylic Paint/finger Nail Polish Sun Sensor

A transparent sun sensor paint/polish was made by adding the monomer 10,12-pentacosadiyneoic acid (PDA) to a clear commercially available nail polish finish (Orly Snap, Orly International, Inc., made with ethyl acetate, butyl acetate, isopropyl alcohol, nitrocellulose, dibutylphthalate, polyvinyl butyral, etocrylene, D&C red #6 barium lake, D&C violet #2) to a final concentration of 100 PDA/ml nail polish finish. The PDA monomer was mixed to clarity. Thin films were made on both a finger nail and a removable adhesive band. The polish dried within minutes. Upon drying the sun sensor polish was exposed to sunlight. A light blue color begins to appear upon mild sun exposure. A deep dark blue color appears upon prolonged exposure. The polish can be easily removed by gentle scraping or with standard finger nail polish remover.

### Example 7

### Multiple Exposure Level Sensor

A multi-exposure level sun sensor was made using polish paints. Transparent sun sensor paint/polish was made by adding the monomer 10,12-pentacosadiyneoic acid (PDA) to a clear commercially available nail polish finish (Orly Snap, Orly International, Inc.) made with ethyl acetate, butyl acetate, isopropyl alcohol, nitrocellulose, dibutylphthalate, polyvinyl butyral, etocrylene, D&C red #6 barium lake, D&C violet #2) to a final concentration of 200 mg PDA/ml nail polish finish, 100 mg PDA/ml nail polish finish, and 50 mg PDA/ml nail polish finish. The PDA monomer was mixed to clarity. Three side-by-side spots were painted with each of the PDA/polish concentrations. The spot containing the lowest concentration of PDA required the longest time to obtain a dark blue color, the middle spot required the second longest exposure time to sunlight, and the spot containing the highest concentration of PDA required the least exposure time to obtain a dark blue appearance. The exposure time required for changing each spot was linear in time in relationship to the concentration of PDA monomer in a specific spot.

The multiple exposure level sensor is ideal for indicating minimal-safe exposure levels for individuals with different exposure tolerances. For example, individuals with extremely sensitive skin should minimize any further exposure when the first and most sensitive spot turned dark blue whereas individuals with a high tolerance to sun exposure should minimize further exposure when the least sensitive spot turns dark blue.

### Example 7

### Stick-on Sun Sensor Tabs for Eye Ware

Transparent tape stickers (¼ inch in diameter circles made with acrylic based adhesive label dye cut and placed on a convenient removal strip) were coated with a solution of 100 mg PDA/ml chloroform. The coating thickness was about 200 microns.

The coating was allowed to dry for 6 hours at room temperature prior to use. The sensor labels can be conveniently placed on any surface to be exposed to ultraviolet light. The sensor labels were placed on the interior of sunglasses to determine the amount of stray and transmitted light that can enter the cavity between the glass lens and the eye. The sensor label is convenient for the individual who wears the sun glass to determine the amount of UV radiation to which the eye is exposed. A simple glance to the location of the sensor allows one to judge instantly or continuously the level of sun exposure within the eye glass-eye cavity. A light blue color begins to appear upon mild sun exposure. A deep dark blue color appears upon prolonged exposure.

### Example 8

### Reverse Message Exposure Sun Sensor

Standard white pressure sensitive labels (Avery ⅝ inch by 1¼ inch Multi-Purpose 05428 S1020) were coated with a solvent monomer solution containing 150 mg 10,12-tricosadiyneoic acid dissolved in chloroform. The solution was dispensed from a felt-tipped marker (Custom Color-T, from Tria). A single coating was applied with single brush strokes and the solvent allowed to dry at room temperature for 5 minutes. White lettering messages were printed on the white background labels using a standard typewriter and typewriter correction film (KO-REC-TYPE, part number 3). White letter messages were typed directly over the pre-coated labels. Messages included: Safe exposure level achieved; Avoid further exposure; Enough sun for today; and the like.

Upon sunlight exposure for 10 minutes (direct sunlight at 12:00 noon) the label's background started turning light blue the words being only partially visible. After 30 minutes exposure, the messages became clearly apparent indicating adequate sun exposure and that further exposure should be avoided.

### Example 9

### Sun Sensor with Color Indicator for Exposure Level

Transparent plastic sun sensor decals with an adhesive backing were prepared using clear ink jet transparency film. A standard ink jet printer (Hewlett Packard model 620 Ink Jet Printer) and a standard software graphics program were used to develop a series of light blue to dark blue squares (¼ inch square). Colon tones were picked which matched the blue tones of the PDA monomer as it becomes irradiated over an hour period in intense sunlight. Five side-by-side blue squares were lined up linearly with the lightest blue square to the left. The words “low exposure” were written above the light blue squares and the words “high exposure” were written above the dark blue squares. A linear strip (⅛ inch wide) of monomer solution was painted directly below the row of blue squares. The strip was coated with a solvent monomer solution containing 150 mg 10,12-tricosadiyneoic acid dissolved in chloroform. The solution was dispensed from a felt-tipped marker (Customer Color-T, from Tria). A single coating was applied with single brush strokes and the solvent allowed to dry at room temperature for 5 minutes.

The sun sensor was placed in bright direct sunlight for a period of 10 to 60 minutes. The monomer strip started to turn light blue after 10 minutes indicating a low level of exposure. During a 60 minute exposure the strip continued to become quantitatively darker indicating that a resulting high level of exposure had been achieved.

### Example 10

### Rub On Sun Sensor Tattoo

Rub on non-permanent tattoos are made using a thin adhesive membrane plastic which have been printed with a monomer solution containing 100 mg 10,12-tricosadiyneoic acid dissolved in a 50:50 mixture of chloroform and acetone. The monomer/solvent mixture was allowed to dry at room temperature for 10 minutes. Designs representing cartoon characters and animals were created in a rubber stamp using commercial vendors as sources. An associated message “Enough Sun for Today” was printed in conjunction with the designs. One inch in diameter label-tattoos were printed with the design/message on lift off paper. The initial appearance of the label-tattoos was clear and transparent. A label-tattoo, which initially appeared colorless, was placed on bare skin and exposed to direct sunlight. Upon application to the skin and exposure to intense sunlight, the design and message became blue. After 10 minutes exposure, the design and message was faintly blue and after 60 minutes exposure, the design became a vivid blue color.

The label-tattoo format is fun, amusing, and can be made attractive for children to wear and encourages continual usage. It also serves as a reminder to parents that their children have been exposed to a certain level of UV irradiation. In addition, the label-tattoo can be used for day care, ski lessons, swimming lessons and other outdoor activities involving sun exposure and children.

It is evident from the above results, that a convenient method for detecting ultraviolet radiation is provided. Anyone can use the devices and compositions for monitoring the level of UV radiation exposure. Of particular importance is the opportunity to protect those people who are sensitive to ultraviolet radiation in a simple convenient manner. The individual can monitor exposure under a wide variety of circumstances using different methods for mounting or administering the photochromic agent containing device or composition to the individual. The various devices and compositions can be readily packaged so as to be easily carried and to be employed at any time the individual is concerned about exposure.

### Example 11

### Packaging Integrating Light/UV Exposure Activation for Revealing Messages and Graphics

Package print integrating light/UV sensitive color development was created by incorporating diacetylenic chromic agents into conventional ink bases and printing processes. Standard solvent based flexographic ink bases and Gravure ink bases were modified to contain 5% solublilized 10,12-tricosadiynoic acid and 1% solubilized 10,12-pentacosadiynoic acid. Both pigmented and non pigmented bases were prepared. Pigments used included white, yellow, red and orange.

Standard coated and uncoated paper stocks were printed on conventional printing presses using 2, 4 and 6 color printing stations. Ink bases containing diacetylenic monomers were loaded at specified printing stations. After printing the packaging material, areas printed with the light/UV sensitive diacetylenic materials were covered and protected by sealing with a clear, but UV blocking acetate laminate. The light/UV sensitive diacetylenic materials remained inactive to light/UV exposure as long as the layer covered the sensitive printed areas. Upon removal of the laminate and exposure to UV light, the diacetylenic monomer containing areas generated an increasingly blue hue with increasing time of exposure.

Graphic images and text were both developed and altered by the color development process. Promotional messages were of particular interest in that the messages were used to indicate contest results and story lines. Graphic images were of interest for product branding and licensed characters.

### Example 12

### Ambient Photo-activated Clock Mechanisms for Telling Duration

Light/UV exposure can be used to activate a duration monitoring process in which a monomeric form of diacetylenic materials turns from an uncolored state to a colored state upon exposure. The color continues to become darker as exposure continues and the degree of coloration and coloration rate of change are dependent on the light/UV intensity. Specifically, for polymerization of diacetylenic monomers, the presence of peak intensity of 254 nanometers begins the polymerization and coloration process.

Photoactivated clock mechanisms can be accomplished using an application means for the diacetylenic monomer such that the deposited material can be exposed directly to UV light at a pre-determined dose. The diacetylenic material is pre-deposited on a substrate and protected from UV light so that clock initiation occurs when the protection is lifted and exposure begins. Alternatively, clock initiation can be accomplished by depositing the diacetylenic monomer from a non-crystalline medium such that the deposition leads to the formation of a crystalline polymerizable form of the diacetylenic material.

In one case, the diacetylenic material was deposited from a solvent based ink onto a paper surface. The monomer deposited on the paper was placed in direct sunlight. The photo-activated mark was made using a felt stamp marker containing N-propyl-icosa-5,7-diynamide monomer dissolved at 100 mg/ml methylene chloride/ethanol 10/4 volume/volume. The solvent ink was introduced into the felt stamp marker reservoir. The mark began to turn color within a minute after placement in the sun. The mark turned progressively darker with exposure time up until 20 minutes by which time the mark had turned a dark blue.

In a second case, a photo-activated clock/label was prepared using a pre-printed label identifying time frames based on coloration densities. The coloration markings were printed to indicate a 12 hour time frame. The numbers 1-12 were graphically rendered to look like a clock face (12 at the upper side and 6 at the lower side). A concentric ring gradient was graphically rendered just below the numbers 1 through 12. The gradient was lightened underneath 1 and continued darker clockwise around the clock until it was the darkest just below the number 12. The gradient was rendered using a blue hue from a low optical density to a high optical density around the ring. The optical densities were calibrated to coincide with the level of color change realized by the light/UV exposure indicating material. The label was circular and measured 1 inch in diameter using a pressure sensitive label stock. The center 0.5 inch of the label remained clear for marking with a light/UV exposure indicating material.

The photo-activated clock/label was started using a felt stamp marker containing N-propyl-icosa-5,7-diynamide monomer dissolved in 100 mg/ml methylene chloride/ethanol 10/4 volume/volume. The solvent ink was introduced into the felt stamp marker reservoir. To begin the counting process, the mark was placed in the center of the 0.5 inch in diameter clear white center. Coloration of the mark was monitored for intensity from the first hour of exposure under ambient room light conditions through 12 hours of ambient room light conditions. The center mark predictably became darker blue as the clock time progressed. Each hour matched the color gradient concentricity around the center mark and at each time referenced on the graphic clock.

The photo-activated clock/label finds use for monitoring indoor and/or outdoor activities. The concentric ring gradient coloration can be more or less intensified depending on the application of use: indoor, outdoor, bright lighting, low lighting, sunny days, cloudy days, and position relative to exposure. Various configurations of the clock/label were developed for specific applications. Medical grade tapes, paper labels, badges, clips and the like are useful substrates for the photo-activated clock/label product.

### Example 13

### Pharmaceutical and Medical Packaging Indicator System Indicating UV Exposure Levels

A UV sensitive indicator was prepared for the purpose of determining the extent of UV exposure of prescription medications. A solvent based ink containing N-propyl-icosa-5,7-diynamide monomer dissolved at 100 mg/ml methylene chloride/ethanol 10/4 volume/volume. The solvent ink was introduced into a felt tip pen marker reservoir. A white paper label (0.5 inch in diameter) was placed on the prescription label of a tamper resistant plastic medication container. For activation, a mark was placed on the paper label with the solvent based pen.

The label mark remained visually transparent as long as the container and medication were kept away from light If the container and mark were exposed to slight stray light (broad spectrum containing low levels of UV light), the mark turned visibly blue over a period of several days. If the mark and container were exposed to direct light (broad spectrum containing low levels of UV light), the mark turned visibly blue within an hour after exposure to light. Intense direct sunlight caused the mark to turn visibly blue within minutes after exposure.

### Example 14

### Plural Light/UV Color Development and Reversible Optical Color State Changing Substrate

Combination color developing/color changing substrates were prepared in which initially a diacetylenic compound was applied in a solvent based carrier mixture to a flexible substrate (e.g. plastic, paper, or pliable metal) and dried. In the monomeric state, the diacetylenic material was colorless. Upon exposure to UV light, the diacetylenic monomer converted to a blue colored form at room temperature.

The polydiacetylenic colored substrate could be mechanically stretched, bent, twisted, poked, displaced or the like such that the polydiacetylenic layer was elongated or compressed. Elongation caused the blue area to reversibly turn a visible reddish hue. Compression caused the blue area to turn reversibly to a deeper darker blue. The torsional reversible color change can be repeated over and over again indefinitely until the substrate material becomes damaged.

A di-diacetylenic monomer amide bis-5,7-hexadecadiyn-ethylenediamide was prepared using a standard acid chloride coupling reaction. The acid chloride of 5,7-hexadecadiyneoic acid was added in a dichloromethane solution to a dichloromethane solution containing 0.5 equivalents of ethylenediamine. The UV sensitive reaction product crystallized into a micro-fine white powdered crystal and was stored in a light-sealed bottle.

A solvent carrier ink was prepared with a 10% suspension of the bis-5,7-hexadecadiyn-ethylenediamide crystalline monomer in acetone and 20% dissolved clear acrylic plastic or soluble cellulosic resin. The ink was brush applied or printed using conventional means on plastic, paper, or pliable metal substrates. After complete drying, the active ink area was exposed to UV light (254 nm) to generate a deep blue color.

Subsequent to color development, the blue area showed a repeatable and reversible color change to a magenta and reddish hue during stretching and pulling. The color change was not due to heat transfer. The color change was due to the strain placed on the chromic agent as transduced through the substrate.

### Example 15

### Plural Light/UV Color Development and Reversible Optical Color State Changing Packaging and Printed Materials

Plastic bottles, shrink wrapped plastic labels, and printed paper packing items were coated with inks containing bis-5,7-hexadecadiyn-ethylenediamide crystalline monomer at a 6% concentration in flexographic and Gravure ink bases as described above.

Graphic and printed items were either printed directly onto a conventional plastic bottle or labeled appropriately such that the active diacetylenic monomer was readily available to a pre-determined UV exposure level. Printed material and graphics were designed to highlight both the photochromic and optical state change properties of the active areas.

Upon UV exposure, printed areas containing the bis-5,7-hexadecadiyn-ethylenediamide crystalline monomer turned blue and generated the desired graphic visualization. Subsequent bending, stretching, twisting, and distorting of the graphic gave the further visual appearance of a blue to magenta or red color change depending on the magnitude of the distortion applied.

Color generating/changing examples such as these appeared particularly important for new interactive packaging, photographs, magazines, books, toys, clothes, sporting goods equipment such as skis, utensils, exercise equipment, and other products where a dual color changing system increases product functionality and interactivity. A variety of food, beverage, and confectionery packaging, lids, and containers are applicable to the interactivity of the color changing properties described.

Alternatively, mediums fully printed with the light/UV active color generating compositions can be over-printed with a transparent light/UV inhibiting coating. The coating can be in the form of a transparent image, graphic or written material. On light UV exposure, a reverse image can be generated on the medium where all of the uncoated areas develop a color, whereas the coated areas remain blank.

### Example 16

### Fluid Mediums for Transient Imaging Effects Based on Light/UV Activated Color Generating Agents in the Mediums

Light/UV initiated images were generated in fluid mediums by adding a homogeneous suspended form of diacetylenic monomers to the fluid. If the monomeric form is in a suspended mirocrystalline state in the fluid medium and the medium is exposed to UV light, the fluid becomes colored in the exposed area. Various fluid mediums were tested including oils, water, solvents, silicones, gels and the like.

An aqueous sonicated form of the diacetylenic monomer, N-ethanol-hexadeca-5,7-diynamide was mixed at a concentration of 0.5% by weight. The particulate monomer crystals formed a translucent solution that was a slightly white pearlescent color. The N-ethanol-hexadeca-5,7-diynamide exhibited rapid reactivity and color development in response to exposure to sun and UV light. The monomer can be processed into a microcrystalline form or liposomal form. Ambient room lighting also initiated color development in less than an hour.

Various colors could be developed from the same solution by increasing or decreasing the temperature of the mixture. Purple and blue hues were developed by exposure to light when the temperature was lowered to below 60° F. Magenta hues were developed when the solution was maintained at room temperature. Red, pink and orange hues were developed upon exposure to light when the solution was maintained at 80° F. or above.

Monomeric polydiacetylenic crystalline solutions provide an interactive photochromic medium for a variety of packaged goods, cosmetic applications, consumer product applications, and industrial applications. Optical changes in the fluid mediums provide for novel optical color changing effects including photochromism, thermochromism, birefringence, fluorescence changes and the like.

Transient graphic images were generated in solutions contained within clear packaging structures. Packaging structure examples includ polyethylene bottles, bags, packets, polypropylene packages and other optically transmissive and inactive barriers. Photomasks were created on the container or package surface such that images could be transiently created within the liquid medium.

The liquid medium containing the crystalline monomeric solution was added in bulk to fill a plastic container. The container surface was patterned using a clear UV blocking resin. The solution was allowed to stand still during exposure to UV light. The area of the solution immediately adjacent to the container wall and directionally inward from the container walls, which were UV transmissive, developed a deep irreversible coloration. Areas where UV light was prohibited from transmitting throughout the container wall remained light and non-colored. The contrasting color boundaries between the UV transmissive areas on the container and UV inhibited areas on the container serve as the basis for development of images within the solution itself. The images were transient and disappeared when the still solution was disturbed.

The image and color developed within the solution deepened in color and distance within the liquid medium with intensity and duration of UV exposure. The color hue of images developed could be tuned based upon the temperature of the liquid medium.

Motions in the patterns formed in the liquid medium could be induced by simple and mild disturbance of the fluid medium. In cases where the liquid medium was contained in a flexible packaging configuration, a transient image initially developed while the solution remained still. The image was distorted by flexing a region on the package and thereby displacing the patterned fluid medium within the package. Release of the distorted package back to its original configuration resulted in restoration of the patterned fluid to its original image. Preservation of the original image depended on diffusion time and flow within the fluid medium.

Images also were formed in frozen fluid mediums when the liquid medium containing the crystalline diacetylenic monomer was frozen in a fixed geometry prior to any exposure to UV light. After freezing, the frozen solution in the container was exposed to UV light and a patterned color developed. The transient color that developed within the fluid medium diffused and was eliminated upon melting the frozen liquid medium.

The liquid medium, containing the crystalline diacetylenic monomer, was also jacketed on the outer exterior of a cuntainer such that a thin layer of the monomeric polydiacetylenic crystalline solution surrounded the internalized container. The solution jacket was made to turn color by exposure to UV and/or sunlight. Images that developed in the jacket were transient; diffusion and mixing of the liquid resulted in diffusion and disappearance of the image. The image could be regenerated by allowing time for the liquid solution to stop moving and then re-exposing the medium to UV and/or sunlight.

### Example 17

### Light Activated Irreversible Color Generation Inks and Printed Materials

Invisible printed graphics, legends, logos, numbers, sweepstakes, tickets, security marks, identification data, bar codes, voting cards, out door security cards, coupons wording, messages and other related printed information can be printed on various substrates using the monomeric form of diacetylenic materials. The printed regions, when exposed to light/UV, appear in their pre-designated patterns.

Standard non-pigmented solvent ink bases were modified to contain 5% solublilized 10,12-tricosadiynoic acid and 1% solubilized 10,12-pentacosadiynoic acid. Ink bases generally were made with cellulosic resins. Inks were formulated according to the printing process applied. The ink viscosity was adjusted for each process by adding the appropriate carrier solvent formulated in the original ink base.

After printing, the UV active regions were protected from premature exposure by overlaying a clear UV protective cellulose layer. The layer was affixed with a transparent adhesive such that the layer, when mounted over the active region, did not interfere with the underlying graphic.

Light/UV active graphics and printing were printed alone or in combination with conventional printing colors to achieve a desired pre-exposure graphic and subsequent post-exposure graphic. The before and after exposure graphics and printing were designed to reinforce an intended communication.

### Example 18

### Light/UV Activated Thermal and Frictional Sensitive Printing Mediums

Paper was ink jet printed with a 150 mg/ml solution of 10,12-tricosadiynoic acid mixed at a 10 to 1 weight ratio with 10,12-pentacosadiynoic acid dissolved in a mixture of ethanol to chloroform 10 to 1 volume to volume. The diacetylenic monomer ink was placed in a cleaned Hewlett Packard ink jet cartridge and printed onto standard 80 pound white paper using a Hewlett Packard desktop printer. Paper sheets were printed with complete coverage using the heaviest print setting.

Printed papers exhibited uniform color formation when evenly exposed to a UV light box (254 nm) for 1-3 minutes or exposed evenly to sunlight for 1 hour. The paper was selected to avoid coatings and whiteners which may interfere with the monomer coating and polymerization process. Polymerization lead to a deep blue hue upon polymerization. The optical density of the blue coat depended on the degree of UV exposure. Increasing the UV exposure level resulted in increasing optical densities of the blue printed surface. Alternatively, overhead transparencies also were used as convenient printing substrates for the polydiacetylenic material.

Once the paper had been UV activated and colored by polymer formation, it was utilized in a variety of subsequent processes for written and graphic communication. Polymer coated paper was further caused to change color using frictional pressures, rubbing, solvent induced color changes, and thermally induced color changes. Written material and graphics were incorporated onto the paper by virtue of the chromic change in state of the blue ordered polydiacetylenic backbone to the disordered red polydiacetylenic form.

Conveniently, a black and white plain paper thermal fax printer (Brother Intellafax) was converted into a color printer by virtue of thermally printing information onto the blue form of a paper sheet. The thermal fax printer was modified to disengage the thermal transfer ribbon. Blue polymer coated sheets were thermally printed in the copy mode or by receiving a fax message by telecommunication. The thermal print head in the thermal fax machine accurately printed the intended information by converting the blue polymer to a bright red in the intended format. Clearly defined images and graphics appeared in red against the blue unaffected areas of the paper.

The blue polymeric coated paper also was used for pencil and pen free writing and drawing. Hand-written or hand-drawn information was imprinted on the polymer coated paper by rubbing an insulative non-metallic point on the polymer region. The rubbing action resulted in marking the page by creating red/orange marks at the point of pressure. Written messages, notes, comments, drawings or the like were conveniently marked in red/orange and contrasted well against the blue polymer background.

In another configuration, plain paper was pre-printed with graphic information using conventional inks. The color hue and intensity were adjusted so that the print was obscured easily using the UV activated diacetylenic overcoat. Over-coating pre-printed graphics provided a convenient means to obscure or hide printed information such that the hidden printed information was revealed subsequently by inducing a color change from the blue form of the polymer to the red form of the polydiacetylenic polymer. The blue polymer was converted to the red polymer to reveal the underlying graphics by frictional rubbing, heating, and/or with treatment with organic solvents.

### Example 19

### Light/UV Exposure Image Generation Mediums

Direct light/UV imaging can be accomplished directly from computers, television, liquid crystal display screens, screens back lit on personal digital assistants, and related flat and tube screen monitors. In addition, image generation can be accomplished using light focused through a lens onto any medium containing a light/UV sensitive photochromic agent. Light/UV sensitive analogs of diacetylenic monomeric materials were deposited on paper as described in Example 18, above. Highly light sensitive analogs including N-ethanol-hexadeca-5,7-diynamide and N-propyl-icosa-5,7-diynamide monomer were used by way of example. Other reversible and irreversible photochromic agents were applied as alternative image generating materials. A wide range of photo-sensitive agents can be applied to direct from screen printing.

For direct-from-screen printing, the screen image must remain stationary for an appropriate duration of time to ensure that the image is captured on the substrate sheet that has been coated with the photochromic agent. Photochromic agents can be selected to match the emitted wavelengths from the image-generating source or the light emitted from the image generating source can be adjusted to match the peak wavelengths suitable for capturing an image onto the photochromic agent medium.

In one case, a printed sheet was prepared as described in Example 18 above. The printed sheet was protected from light exposure prior to use by storing it in an optically opaque folder. The sheet was laminated along one edge with a UV protective yet visibly transparent sheet which covered the entire printed sheet. The paper was printed using N-ethanol-hexadeca-5,7-diynamide in a non-polymerized ink form.

For exposure, a laptop computer screen was loaded with a striped pattern and then the screen back light was turned off until after the monomer printed paper had been pressed against the screen. The paper was secured and flattened against the screen, then the screen back light was illuminated in order to expose a pattern on the printed paper. Exposure was continued for 5 minutes. After exposure, the paper was removed and laminated with a transparent protective sheet. The inverse of the striped image on the screen had been visibly imaged onto the printed paper. A variety of direct-from-screen images were developed and printed using the process described.

### Example 20

### Light/UV Exposure and Limited Lifetime Signs

Commercial signs, billboards, advertisements, warning signs, collateral material and other outdoor signage can be activated to reveal information upon light/UV exposure, retain the graphic or printed material for a period time, and finally photo-degrade the graphic or printed material after a specified duration to blank out what had originally been printed. Light/UV limited lifetime signage provides novel attractive features to printing in that commercial signs can be made to expire, thereby eliminating the need for example for periodic removal when a lease is up.

Outdoor signs were prepared by printing N-ethanol-hexadeca-5,7-diynamide at low concentrations using printing methods described in Example 18. A sign was printed with the monomer as described and then placed in direct sunlight on day one. The printed information became visible immediately upon exposure for 2 minutes. The sign was attached to a stationary billboard such that sunlight would shine directly on the sign each day. The sign was monitored for color degradation daily. Noticeable lightening of the print occurred after one week of exposure. The print was completely photobleached and eliminated after two weeks of exposure. No visible printed information remained.

The duration of print longevity and onset of photo-degradation can be extended or reduced depending on the amount of light/UV exposure intensity, the amount of chromic agent deposited on the substrate, and the use of light/UV inhibitors added to the ink composition.

### Example 21

### Light/UV Active Cosmetic, Hair, Skin, and Personal Care Additives

Additive compositions containing light/UV active photochromic agents were prepared by making powered crystalline formulations of the agents. The microparticulate formulations are added directly to a cosmetic, hair care, skin care or other personal care item. Addition of the agent formulation to a personal care item provides for novel photo-chromic coloration and color change properties for the item. Leave-in hair conditioners, hair spray, hair gels, lotions, ointments, message lotions, skin powders, sunscreen lotions, eye shadow, foundations, rouge, cosmetic bases, body oils, mascara, lipstick, medical ointments, and related personal care products were treated with powdered crystalline diacetylenic monomeric compositions.

N-ethanol-hexadeca-5,7-diynamide, 10,12-tricosadiynoic acid, 10,12-pentacosadiynoic acid, bis-5,7-hexadecadiyn-ethylenediamide, N-propyl-icosa-5,7-diynamide and related chain length diacetylenic monomers were powderized neat/dry using a hand-held high speed electric coffee grinder. Particle sizes were measured by particle sizing to be approximately 60 microns in diameter. Monomer powders were kept out of light to maintain their colorless appearance. Water pastes also were prepared by admixing water with the powdered monomers. The paste was made by continuous hand mixing (70% by weight water).

Monomer additive compositions were added directly to personal care item matrices. Oil-based matrices were treated with dry powdered monomeric material and water-based matrices were treated with the water-based monomer pastes. The monomeric additive compositions were added at concentration ranges from 1 to 10% by weight of the personal care item matrices. The amount of monomeric materials added depended upon the opacity of the matrices being added. Highly opaque matrices typically required higher concentrations for more complete visualization when the monomer was converted to the colored polymeric form. Less opaque more translucent matrices required lower concentrations of the monomer additive since the final colored polymeric material was less obscured.

Personal care items were applied to skin or hair depending upon their intended application of use. After normal application and sun exposure, the applied formulation turned color as a result of the polymerization reaction in the diacetylenic additive. Color changes include blue hues for higher thermochromic transition polydiacetylene, magenta hues for medium thermochromic transition polydiacetylenes, and orange/red hues for low thermochromic transition temperature polydiacetylenes. The diacetylenic composition is selected depending on the color hue desired for a particular application.

Personal care items integrating the light/UV chromic material where the material became colored upon exposure can subsequently and additively undergo a second color change based on heating or frictional forces. By way of example, hair spray containing N-propyl-icosa-5,7-diynamide causes sprayed hair to turn blue in sunlight. Subsequent blow drying with hot air will cause the blue color to turn red reversibly. The red color will return to a blue color upon cooling back to room temperature.

### Example 22

### Light/UV Activated Sensing Agents Integrated in Sunscreens

In the case of sunscreen sun sensor additives, larger flake-like monomer additive compositions are desirable as added light/UV indication agents. The larger particle size is desirable since the presence of visible color changing particles provides for a means to calibrate the presence and effectiveness of the applied sunscreen. Since monomeric diacetylenic crystalline materials turn color in the presence of UV/sun light, the material acts as an integrated sensor to judge exposure levels.

SPF 30 sunscreen lotion was admixed with 1% by weight 10,12-pentacosadiynoic acid powder (500 micron particle size). After uniform mixing, the sun sensing sunscreen was applied to skin. The uncolored monomeric crystal particles were visible only by very careful inspection and were not visible from more than 12 inches away. After exposure to the recommended 60 minutes of sunlight (post peak hour), the crystal particles turned lightly blue and were visible if their presence was known. The light blue polymeric particles were not obviously visible to bystanders. The sun sensing particles were accurate indicators that it was time to apply another coating of sunscreen.

The polydiacetylenic material was further made to change color by the frictional forces applied to skin by the second application of sunscreen. During the second application and rubbing, the blue polydiacetylenic particles became irreversibly light red and disappeared against the skin color. The disappearance of the original particles provided a means for re-initiation of color formation from particles delivered in the second sunscreen application. The processes can be repeated numerous times.

### Example 23

### Light/UV Activated Pen for Cosmetic Applications

Felt tip pens for marking substrates with solvated monomeric diacetylenic materials can be used for cosmetic applications such as creating UV/sun sensitive markings on skin, entertainment applications such as writing which becomes visible from a transparent mark when exposed to sunlight, and various outdoor applications where it is of interest to determine sun/UV levels.

By way of example, a felt tip pen marker was prepared using N-propyl-icosa-5,7-diynamide monomer dissolved at 100 mg/ml in methylene chloride/ethanol 10/4 volume/volume. The solvent ink was introduced into the felt marker reservoir. The pen was used to mark various surfaces including skin, paper, wood, and other surfaces that were exposed subsequently to sunlight. A blue coloration began to appear immediately within a minute of sun/UV exposure. The mark continued to darken overtime and became dark within 5 minutes.

### Example 24

### Light/UV Integrating Sensor for Determining Light Exposure Levels for Vegetation and Planting

Light/UV indicating devices were used to determine optimal light levels for planting indoor, outdoor, and greenhouse plants and vegetables. Wood, plastic, paper, and other composite materials served as useful marker substrates for adhering a light/UV sensitive indicator for monitoring accommodative appropriate exposure levels for particular plants. The light/UV indicating material can be calibrated to turn color based upon light levels from low level light requiring plants to high level light requiring plants.

Plant sensors were prepared by applying a monomeric diacetylenic material on a suitable substrate and further fitting a UV protection layer over the light reactive area until the sensor was ready to be used. Monomer type and level were pre-determined depending on the optimal light level to be monitored. Sensors were calibrated to change color over periods long enough to determine daily light levels at a particular location intended for planting.

By way of example, an ink was prepared with a 150 mg/ml solution of 10,12-tricosadiynoic acid mixed at a 10 to 1 weight ratio with 10,12-pentacosadiynoic acid dissolved in a mixture of ethanol to chloroform 10 to 1 volume to volume. The ink was printed onto the blunt end of a polystyrene plastic stake in a 0.5 inch square. The stake was 0.5 millimeter thick, 0.5 inch wide and 3.5 inches long. The stake was pointed at one end to assist in inserting it into soil or other surfaces. The printed square was covered with an adhesive light blocking film that inhibited color generation until the plant sensor was ready for use. The plant sensor photo-active square printed area was calibrated to turn a deep blue over a one day period in a 50% shaded area.

In use, the plant sensor was inserted into the soil of a potted geranium. The plant was placed in a yard region anticipated to have the correct light level for the plant type.

The sensor was activated by removal of the light blocking film. Color development was monitored throughout the day. By the end of a full day, the printed square turned a deep blue confirming that the yard location had ideal lighting for the plant type.

Alternative configurations for plant sensors included sensors integrated directly into planters, pots, tags to be attached to plants, applicators for applying the light/UV material directly onto leaves or plant stems, various wood and metal stakes, and the like.

The specific sensor configuration desired depends on the type of plant, location for planting, routine for use, and other parameters which impact performance.

Prior to planting, the sensor was placed in the intended location with the sensing region pointing in toward the expected direction of the sun or indoor lighting. The sensor was monitored for color formation at certain time frames. As exposure occurred, the monomer started to polymerize and became increasingly darker. Calibration colors were used as references in order to judge and compare the color formation in the diacetylenic material to an anticipated color level predicted for the desired lighting level.

### Example 25

### Polymerizable Diacetylenic Compounds Covalently and Non-covalently Coupled with Light Isomerising Compounds

The photo-polymerizable and optical properties of diacetyhlenic polydiacetylenic materials were modulated by integrating compounds that undergo photo-induced isomerization when exposed to specific light/UV levels. Cis/trans isomerizing compounds such as diazo moieties find use as optical conformational change inductors. When attached or directly associated with diacetylenic or polydiacetylenic materials, cis/trans isomerizing compounds were found to transduce subsequent conformational changes in the orientation of the polydiacetylenic composition. The isomerization process in the isomerizing compound can be used to reversibly or irreversibly trigger a color change in the polydiacetylenic materials.

The process was used by way of example to create integrating light/UV induced graphic changes where the changes result in a visible graphic transformation from one image to another. Self-transitioning graphics find use as two-dimensional frame shifts or sequential images. The resulting frame shifts can be used to create the effect of motion in a stationary two-dimensional graphic without the use of optical interference or prisms such as are used in holograms. 4,4′-diaminoazobenzene was chemically coupled at both free amino positions with 5,7- hexadecadiynoic acid groups to form dual amide linkages symmetrically opposite to each other. Coupling was accomplished by forming the acid chloride of 5,7-hexadecadiynoic acid and reacting two molar equivalents of the acid chloride with one molar equivalent of 4,4′-diaminoazobenzene. The finished product was light/UV sensitive and can be polymerized to form the polydiacetylenic structure. The polymer form has a general blue intensity which can be wave length shifted by optically inducing the cis/trans isomerization in the azobenzene moiety.

Alternatively, 4,4′-diaminoazobenzene was added directly to a mixture containing diacetylenic monomers such as bis-5,7-hexadecadiyn-ethylenediamide and N-propyl-icosa-5,7-diynamide. When the compounds were co-crystallized or compounded so that they were in molecular contact with each other, the optical characteristics of the polydiacetylenic backbone were modulated by the cis/trans isomerization properties of the azobenzene compound present.

### Example 26

### Novel Silicon Rubber Composites with Multipuroose Function for Skin for Coating

Light/UV color sensitive agents were combined with thermostat, elastic, thermoelastic, silicon rubber compositions, and reologically responsive materials to create thermo-responsive and photo-color developing compositions. The temperatures for thermal responsiveness of the carrier composition were combined with the light/UV responsiveness of UV active agents such as diacetylenic materials and thermochromicity can be adjusted to be compatible or the like. Combining the crystalline and amorphous characteristics of thermally responsive polymeric systems with the light/UV responsive characteristics of photochromic agents provides for a wide range of materials with a multiplicity of independent functionalities.

By way of example, N-ethanol-hexadeca-5,7-diynamide, 10,12-tricosadiynoic acid, 10,12-pentacosadiynoic acid, bis-5,7-hexadecadiyn-ethylenediamide, N-propyl-icosa-5,7-diynamide and related chain length diacetylenic monomers were powderized neat/dry using a hand-held high speed electric coffee grinder as described in earlier examples. Monomer powders were kept out of light to maintain their colorless appearance.

Powdered monomeric diacetylenic materials were blended with thermally mechanically responsive/non-photoactive materials such as block co-polymers of acrylic acids, acrylic acid esters, vinyl, polyacrylonitriles, polyethylene, polypropylenes, functional co-polymeric compositions, and the like to create dual thermotransitioning/photo-responsive compositions.

The thermal transition temperature of the polymeric non-photoactive materials can be selected for particular applications in combination with the light/UV responsiveness of the diacetylenic compositions. The combined nature and characteristics were integrated depending on the application of interest. Examples include materials with a predictable melting point combined with a predictable color change at the predicted melting point. Alternatively, the material can have a predictable gas permeability at one temperature and indicate a change in permeability with a predictable color change.

It is evident from the above results, that a convenient method for using light/ultraviolet radiation for activation of photochromic agents for a variety of purposes is provided. The devices and compositions can be used for monitoring the level of UV radiation exposure, for revealing messages and graphics on a number of different substrates and for image generation in media including liquids and solids. Of particular importance is the opportunity to protect those people who are sensitive to ultraviolet radiation in a simple convenient manner. The photochromic agents also find use with other agents such as thermochromic agents. The various devices and compositions can be readily packaged so as to be easily carried and to be employed at any time the individual is concerned about exposure.

All publications and patent applications mentioned in this specification are herein incorporated by reference to the same extent as if each individual publication or patent application was specifically and individually indicated to be incorporated by reference.

The invention now being fully described, it will be apparent to one of ordinary skill in the art that many changes and modifications can be made thereto without departing from the spirit or scope of the appended claims.

1-2. (canceled)

3. A composition for measuring cumulative light or UV exposure, wherein said composition comprises:
a photochromic agent in an amount sufficient to provide a detectable color change upon exposure to at least one of light and UV radiation, and wherein said composition is formulated for use on hair, nails or skin.

4. The composition of claim 3, wherein said composition is a skin care composition formulated as a lotion.

5. The composition of claim 3, wherein said composition includes a sunscreen.

6. The composition of claim 3, wherein said composition is a skin care composition formulated as a balm.

7. The composition of claim 6, wherein said balm is a lip balm.

8. The composition of claim 3, wherein said composition is a cosmetic composition.

9. The composition of claim 8, wherein said composition is a skin powder, eye shadow, foundation, rouge, cosmetic base, mascara or lipstick.

10. The composition of claim 3, wherein said composition is a medical ointment, massage lotion or massage oil.

11. The composition of claim 3, wherein said composition is a hair care composition formulated as a leave in conditioner, hair spray or hair gel.

12. The composition of claim 3, wherein said composition is a nail care composition formulated as a nail paint or polish.

13. The composition of claim 3, wherein said detectable color change is reversible.

14. The composition of claim 3, wherein said photochromic agent is a diacetylenic agent.
